(19) United States
(12) Patent Application Publication
(10) Pub. No.: US 2025/0322913 A1
(43) Pub. Date: Oct. 16, 2025

(54) Systems and Methods for Enhanced Acquisition of Mass Spectrometry Data

(71) Applicant: MOBILion Systems, Inc., Chadds Ford, PA (US)

(72) Inventors: John Daniel DeBord, Chadds Ford, PA (US); Ding Zhang, Wilmington, DE (US)

(73) Assignee: MOBILion Systems, Inc., Chadds Ford, PA (US)

(21) Appl. No.: 19/175,525

(22) Filed: Apr. 10, 2025

Related U.S. Application Data
(60) Provisional application No. 63/632,942, filed on Apr. 11, 2024.

(51) Int. Cl. G16B 40/10; G01N 27/623

(52) U.S. Cl. CPC G16B 40/10; G01N 27/623

(57) ABSTRACT

A method for performing real-time data binning of spectrometer data includes receiving, at a digitizer including hardware and software and from a mass spectrometer, first scan data associated with a first scan from a plurality of scans. The method also includes receiving, at the digitizer and from the mass spectrometer, second scan data associated with a second scan from the plurality of scans. The method also includes combining (e.g., summing) the first scan data and the second scan data via the digitizer, to produce first summed scan data. The method also includes modifying, via the hardware of the digitizer, the first summed scan data based on a predefined intensity threshold, to produce second summed data. The method also includes analyzing, via the digitizer, the second summed data to identify a composition of a sample associated with the plurality of scans.

### CROSS-REFERENCE TO RELATED APPLICATIONS

The present application claims the benefit of priority to U.S. Provisional Patent Application No. 63/632,942 filed on Apr. 11, 2024, the entire content of which is incorporated herein by reference.

### FIELD

The present disclosure relates to mass spectrometry, and more specifically, to the real-time processing, including summing and subsequent intensity thresholding, of mass spectrometer data to improve the sensitivity and signal to noise ratio associated with mass spectrometry data.

### BACKGROUND

Most mass spectrometry systems include a digitizer either in the form of a time-to-digital converter (TDC) or an analog-to-digital converter (ADC). A digitizer is a device that receives, digitally processes, and digitally records information about an electronic signal. In the case of mass spectrometry, the digitizer records signals that can be correlated with the mass to charge ratio (m/z) of ions of interest.

### SUMMARY

In some embodiments, a method for performing real-time data binning and enhanced acquisition of spectrometer data includes receiving, at a digitizer and from an ion mobility mass spectrometry (IM-MS) system, first scan data associated with a first scan from a plurality of scans. The method also includes receiving, at the digitizer and from the IM-MS system, second scan data associated with a second scan from the plurality of scans. The method also includes combining (e.g., summing) the first scan data and the second scan data via the digitizer, to produce first summed scan data. The method also includes modifying, via the hardware of the digitizer, the first summed scan data to produce second summed data that includes only data points above a predefined intensity threshold. The method also includes causing transmission of the second summed data to a remote compute device for identification of a composition of a sample associated with the plurality of scans.

In some embodiments, a method for performing real-time data binning of spectrometry data includes receiving, at a digitizer and from a mass spectrometer of an IM-MS system, spectrometer data associated with a plurality of scans. The method also includes summing, via software of the digitizer, a plurality of subsets of data from the spectrometer data, to produce summed scan data. The method also includes generating, via the digitizer, a representation of a mass spectrum based on the summed scan data and a predefined intensity threshold, e.g., such that data values below the predefined intensity threshold are omitted. The method also includes causing transmission of the representation of the mass spectrum to a remote compute device for identification of a composition of a sample associated with the spectrometer data. A number of subsets of data in the plurality of subsets of data may be selected based on a predefined binning number. Generating the mass spectrum can be performed at least in part using hardware (e.g., one or more field-programmable gate arrays (FPGAs)) of the digitizer.

In some embodiments, a non-transitory processor-readable medium stores instructions that, when executed by a processor, cause the processor to receive, from a mass spectrometer of an IM-MS system, spectrometer data associated with a plurality of scans. The non-transitory processor-readable medium also stores instructions that, when executed by a processor, cause the processor to combine a plurality of subsets of data from the spectrometer data, to produce summed scan data. The non-transitory processor-readable medium also stores instructions that, when executed by a processor, cause the processor to identify a representation of a mass spectrum based on the summed scan data and a predefined intensity threshold, the mass spectrum including only data points having values below the predefined intensity threshold.

In some embodiments, an apparatus comprises (1) an ion mobility mass spectrometry (IM-MS) system including an ion mobility separation device, and (2) a first compute device. The first compute device is operably coupled to the IM-MS system, and includes a processor and a memory. The memory stores instructions that, when executed by the processor, cause the processor to receive, from the IM-MS system (e.g., from a mass spectrometer thereof), spectrometer data associated with a plurality of scans. The memory also stores instructions that, when executed by the processor, cause the processor to generate a sum of a plurality of subsets of data from the spectrometer data, to produce summed scan data. The memory also stores instructions that, when executed by the processor, cause the processor to generate a representation of a mass spectrum based on the summed scan data with a predefined intensity threshold such that data values below the predefined intensity threshold are omitted from memory (e.g., by not storing them in memory in the first instance, or by deleting the data values below the predefined intensity from the memory). The memory also stores instructions that, when executed by the processor, cause the processor to cause transmission of the representation of the mass spectrum to a second compute device different from the first compute device.

### BRIEF DESCRIPTION OF THE DRAWINGS

FIG. 1 is a diagram showing an example of a Structures for Lossless Ion Manipulation (“SLIM”) mass spectrometry (“MS”) (collectively, “SLIM-MS”) system architecture, according to some embodiments.

FIG. 2 is a diagram showing an example data flow in a digitizer, according to some embodiments.

FIGS. 3A-3B show plots illustrating real-time binning and intensity thresholding of spectrometry data, according to some embodiments.

FIG. 4 is a diagram showing how signal and noise are impacted by real-time binning and intensity thresholding of spectrometry data, according to some embodiments.

FIG. 5 is a diagram demonstrating the impact of real-time binning on ion mobility plots of counts versus arrival time, according to some embodiments.

FIG. 6 is a flow diagram showing a first method for performing real-time binning of spectrometer data, according to some embodiments.

FIG. 7 is a flow diagram showing a second method for performing real-time data binning of spectrometer data, according to some embodiments.

### DETAILED DESCRIPTION

Modern time of flight (TOF) mass spectrometer systems rely on high speed analog to digital converter (ADC) digitizers which register signals from an electron multiplier detection system, where the amplitudes of the signals correlate with the numbers of ions striking the detector at a given point in time. Because individual TOF mass spectral scans, or transients, typically contain limited ions at a given m/z detection channel (<100 ions), many TOF transients (100-10,000) are typically summed to produce a spectral signal with sufficient spectral quality to accurately represent the relative abundances present within the associated sample. A notable exception to this approach can be found in the case of ion mobility-mass spectrometry systems, where individual TOF transients may be recorded to ensure proper acquisition of ion mobility peaks, which arrive at the MS detector over a limited number of transients. In this case, the known methodology of summing hundreds of transients would obscure the arrival time information as to when certain ion mobility peaks arrived at the TOF mass spectrometer. For the reasons stated above, ion mobility-mass spectrometry data can suffer from low signal-to-noise spectra. The present disclosure describes a system and method for enhancing the acquisition of mass spectrometry data by summing lower numbers of TOF transients prior to eliminating data points below a predetermined threshold. This approach increases the signal level for each summed spectrum while also reducing data size and eliminating noise.

In some known mass spectrometry systems, noise (from electrical or background ion/electron noise sources) can be imparted to/aliased onto output signals (e.g., ion mobility data, time of flight mass spectrum data), and in turn can be present in the digitized spectrum/spectra (e.g., generated using analog-to-digital converter(s) (ADCs)) and the associated final or histogrammed mass spectrum/spectra. With noise present, relatively weak/low intensity ion signals can be obscured or not detected. While some known systems seek to address these noise issues via post-processing (e.g., further processing the data in an attempt to remove/reduce noise after the data has otherwise been initially collected and saved into a data file), such post-processing typically does not improve the sensitivity or signal to noise ratio of mass spectra to a satisfactory degree.

One or more embodiments of the present disclosure address the noise issues discussed above using a process referred to herein as “real-time binning” (“RTB”). RTB can be performed in the context of (e.g., concurrently with) real-time acquisition, and can involve at least two steps: (1) a first step in which data associated with multiple scans are summed, “binned,” or otherwise combined, and subsequently, and (2) an intensity thresholding step performed based on the summed, binned, or otherwise combined data. RTB facilitates one or more of: improved sensitivity of a mass spectrometry system, improved signal to noise ratio of the mass spectrometry system, a reduction in the number of scans and/or data size for processing via software of the mass spectrometry system, a smoothing of mobilograms generated by the mass spectrometry system (e.g., by decreasing the granularity in ion mobility), and a reduction in file size associated with output files of the mass spectrometry system. As used herein, a “mobilogram” (also referred to herein as a mobiligram) refers to a plot of intensity data along an arrival time axis having units of milliseconds, wherein ions separated by size and charge appear as distinct peaks of signal intensity, also known as arrival time distributions. This arrival time axis of the mobilogram may also be calibrated to alternate units of mobility (cm²/Vs) or collision cross section (Å²). A mobilogram can be generated based on a single ion mobility frame or multiple ion mobility frames, where a frame is represented by a fixed number of TOF transients recorded to capture the arrival time range of interest.

RTB can be implemented, for example, in a digitizer that is included in a mass spectrometry system (e.g., a structures for lossless ion manipulation (“SLIM”) mass spectrometry (“MS”) (collectively, “SLIM-MS”) system). Example details of a SLIM MS system compatible with systems and methods set forth herein can be found, by way of example, in U.S. Pat. No. 10,317,364, titled “Method and Apparatus for Ion Mobility Separations Utilizing Alternating Current Waveforms” and issued on Jun. 11, 2019, the content of which is incorporated by reference herein in its entirety for all purposes. One or more aspects of RTB can be implemented in firmware and/or software of the digitizer (e.g., such that no hardware changes are made relative to the digitizer prior to the RTB being implemented). Alternatively or in addition, one or more aspects of RTB can be implemented within hardware of the digitizer (e.g., in a field-programmable gate array (FPGA)). Alternatively or in addition, RTB can be implemented as a selectable (e.g., user-selectable) mode (RTB mode) of the digitizer, optionally in combination with one or more other modes, such as zero summation (“ZS”) mode and/or real-time averaging (“AVG”) mode. As such, RTB may be switched “on” or “off” during operation of the mass spectrometry system, as desired. The RTB mode may be configurable (e.g., via a graphical user interface (“GUI”) of the mass spectrometry system, and by a user), such that the RTB is performed using a configuration from a plurality of possible configurations. These configurations can include variations in a number of MS scans to be binned and/or a level of thresholding to be applied during the subsequent intensity threshold processing. In some implementations, RTB is part of or compatible with a continuous simultaneous acquisition and readout, with triggers (CST), capability of the digitizer, whereby data can be transferred to a display for real time visualization of the streaming data.

Although described above as residing in a digitizer, RTB, in other embodiments, can be implemented in firmware and/or software that resides in a compute device that is not a digitizer, but that is included in, operably coupled to, or in communication with a SLIM-MS system that includes a digitizer. Similar to the above, in such embodiments, RTB can be implemented as a selectable (e.g., via the compute device) mode, optionally in combination with one or more other modes, such as zero summation (“ZS”) mode and/or real-time averaging (“AVG”) mode, and RTB may be switched “on” or “off” during operation of the mass spectrometry system. The RTB mode may be configurable (e.g., via a graphical user interface (“GUI”) of the compute device, and by a user), such that the RTB is performed using a configuration from a plurality of possible configurations. These configurations can include variations in a number of MS scans to be binned and/or a level of thresholding to be applied during the subsequent intensity thresholding.

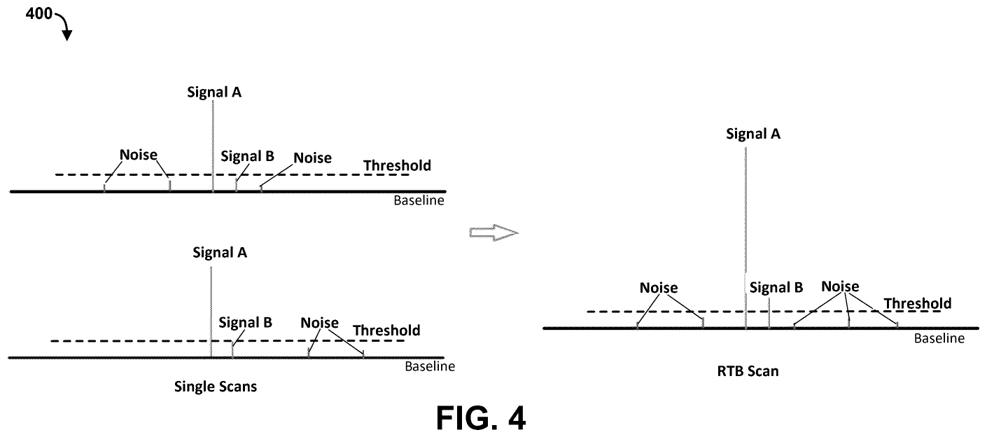

The first step of the RTB process can include the combining (also referred to herein as “binning” or “summing”) of data from each of a plurality of scans, to form a new, combined/composite scan, as shown and further described below with respect to FIG. 4. The combining can include adding together the data from each of the plurality of scans. This “addition” can include the addition of the values of a dependent variable (e.g., counts, relative abundance (%), relative intensity, etc.) for each value of an associated independent variable (e.g., arrival time, mass divided by charge number (m/z) in atomic mass units (amu), etc.). The binning step of the RTB process can be performed based on a “binning number,” which may be user-defined (e.g., via a GUI) and indicates a number of discrete scans to be combined. In some cases, the binning number may be selected or modified (e.g., reduced) based on one or more of the following considerations: tolerance for loss of raw single scan information, a desired resolution of an ion mobility spectrometry (IMS) mobilogram, an expected or actual number of bits involved in representing a summed count, etc.

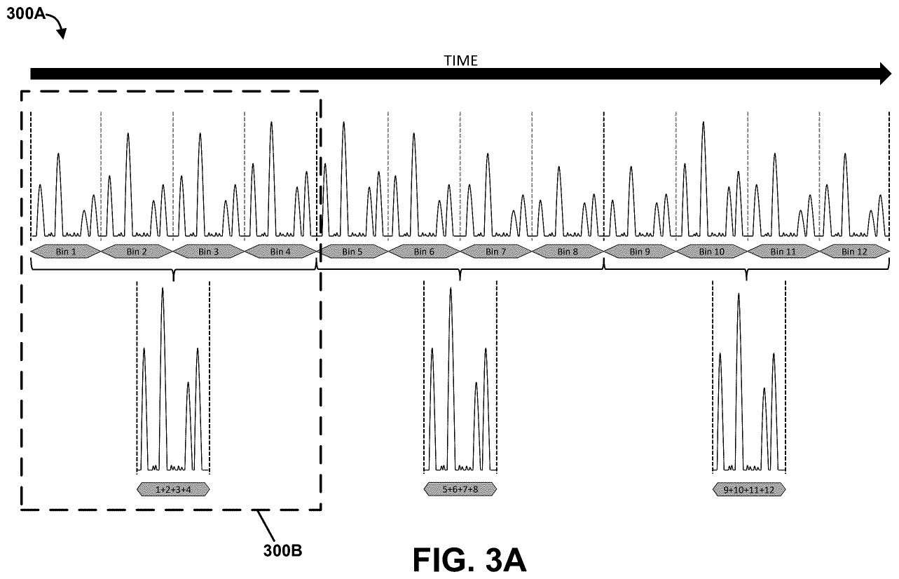

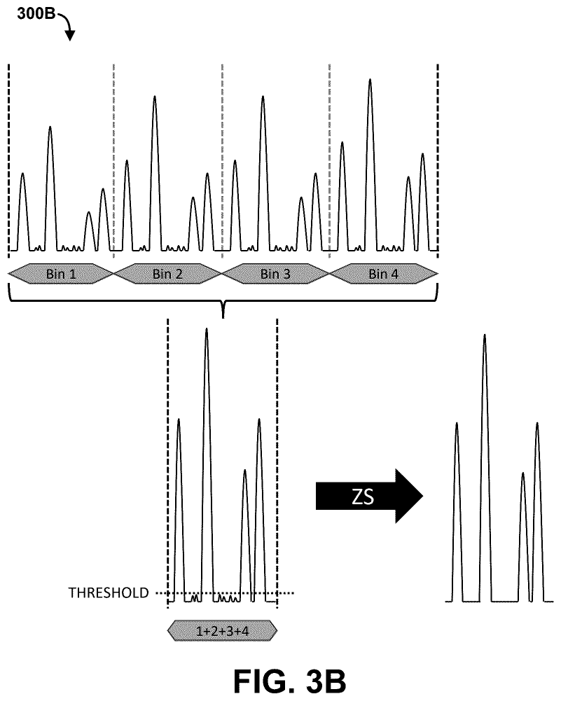

In some embodiments, intensity thresholding is performed based on a predefined (e.g., user-defined) threshold, and only data points above the predefined threshold are recorded, as shown and further described below with respect to FIGS. 3A-3B and 4. The threshold may be selected or adjusted, for example, based on one or more requirements or parameters of a mass spectrometry system (e.g., of one or more data acquisition systems thereof) to operate. For example, there may be a limit to the amount of data that can be processed by a given data acquisition card while maintaining a desired processing rate (e.g., ˜2 GHZ rate, 14-bit), and thus a relatively higher threshold may be desired in some instances. Alternatively or in addition, the threshold may be selected or adjusted based on a relative importance of data, e.g., such that data of interest (of relatively higher importance) is preserved, and data that is not of interest (of relatively lower importance) is not preserved/is discarded. This threshold can be set at a certain level to ensure most single ion detection events are recorded, given that the signal level produced by individual ion detection events is based on a pulse height distribution and not a single discrete value.

In some embodiments, an RTB process is associated with only one ion collection region, only one anode (where the amplified electrons strike during operation), only one digitizer, only one ion detector, and/or only one processing path. Alternatively or in addition, in some embodiments, RTB does not include combining multiple frames and can thereby be applied to a single IMS experimental cycle.

One or more RTB embodiments of the present disclosure enhance the acquisition of mass spectrometry data by summing lower numbers of TOF transients prior to eliminating data points below a predetermined threshold. Such an approach(es) increases the signal level for each summed spectrum while also reducing data size and eliminating noise. RTB facilitates one or more of: improved sensitivity of a mass spectrometry system, improved signal to noise ratio of the mass spectrometry system, a reduction in the number of scans and/or data size for processing via software of the mass spectrometry system, a smoothing of mobilograms generated by the mass spectrometry system (e.g., by decreasing the granularity in ion mobility), and a reduction in file size associated with output files of the mass spectrometry system.

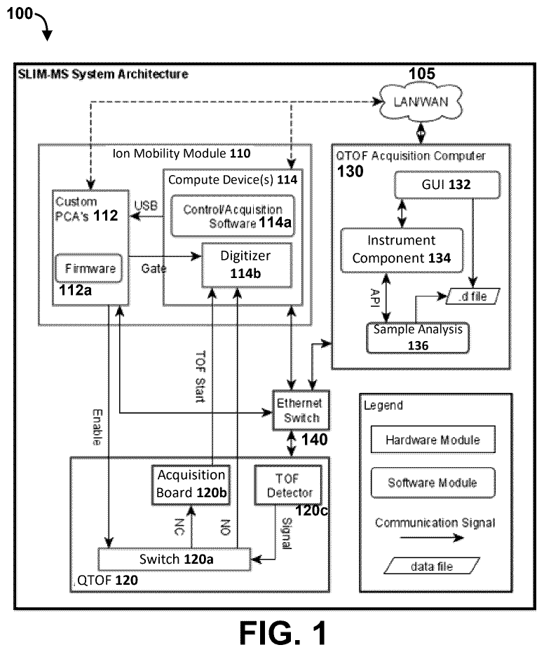

FIG. 1 is a diagram showing an example of a SLIM-MS system architecture, configured to perform high-resolution ion mobility mass spectrometry, according to some embodiments. As shown in FIG. 1, a SLIM-MS system 100 includes an ion mobility module 110, a quadrupole time-of-flight (QTOF) mass spectrometer 120 (e.g., an Agilent 6545, 6545XT, 6546, etc.), a QTOF acquisition computer 130, and an Ethernet switch 140. The ion mobility module 110 (e.g., a SLIM device, examples of which can be found, by way of example only, in U.S. Pat. No. 10,317,364, the entirety of which is herein incorporated by reference above) includes one or more printed circuit board assemblies (PCAs) 112 operably coupled (e.g., via a universal serial bus (USB) or Ethernet interface) to one or more compute devices 114 (e.g., mini-ITX computer(s) or other compute device(s)). Each of the one or more compute devices 114 includes control/data acquisition software 114a and a digitizer 114b (e.g., Aqiris SA220P or similar digitizer) implemented in hardware and software (e.g., including firmware). Each of the one or more printed circuit board assemblies (PCAs) 112 includes firmware 112a. At least one of the PCAs 112 can be configured to transport ions and/or to perform high-resolution ion mobility separation (e.g., using traveling wave separation). Example PCAs and related details compatible with some embodiments of the present disclosure can be found, by way of example, in U.S. Patent Application Publication Number 2021/0382006, the contents of which are hereby incorporated by reference in their entirety for all purposes. Although shown and described in FIG. 1 as specifically including a QTOF mass spectrometer (e.g., an Agilent 6545, 6545XT, 6546, etc.), the present disclosure also contemplates other implementations in which one or more other models of QTOFs and/or one or more TOF mass spectrometers that do not include a quadrupole analyzer are alternatively used.

The QTOF 120 includes a switch 120a (e.g., a subminiature version A (SMA) switch), an acquisition printed circuit board 120b, and a time of flight detector 120c. The QTOF acquisition computer 130 includes a processor and a memory storing a graphical user interface (GUI) 132, an instrument software component 134, and sample analysis software 136 (e.g., Agilent Mass Hunter software), each including instructions executable by the processor. The sample analysis software 136 can be configured to generate/output files such as “.d” files, which in turn may be displayed via the GUI 132. The instrument software component 134 and the sample analysis software 136 can communicate via an application programming interface (API). The ion mobility module 110 is in operable communication, via a communications network (local area network (LAN)/wide area network (WAN)) 105, with the QTOF acquisition computer 130. Additionally, the ion mobility module 110 is operably coupled to the QTOF 120 directly (e.g., hard-wired and/or via a wireless communication channel(s)) and/or via the Ethernet switch 140. Additionally, the QTOF acquisition computer 130 is operably coupled to the QTOF 120 via the Ethernet switch 140. As shown in FIG. 1, during operation of the SLIM-MS system 100, an “enable” signal can be sent from the custom PCA(s) 112 (e.g., via the firmware 112a) to the switch 120a of the QTOF 120, a gating signal can be sent from the custom PCA(s) 112 to the digitizer 114b, and a “TOF start” signal can be sent from the acquisition board 120b of the QTOF 120 to the digitizer 114b of the compute device(s) 114 of the ion mobility module 110, to initiate spectrometry measurements and/or data capture and processing. Alternatively, a trigger signal can be generated by monitoring/converting a TOF pusher signal which initiates the mass spectral analysis to synchronize acquisition with the digitizer.

The digitizer 114b can be configured to perform real-time signal processing, optionally in combination with real-time linearity compensation and trigger timing and/or channel alignment. The digitizer 114b can include one or more memories (e.g., DDR4 SDRAM memory), a trigger time interpolator, a reference clock, one or more direct current (DC) front-ends coupled to input terminals, and one or more FPGAs each including a clock, input/output control, PCT streaming capability, and one or more internal memories with DDR4 control (not shown in FIG. 1).

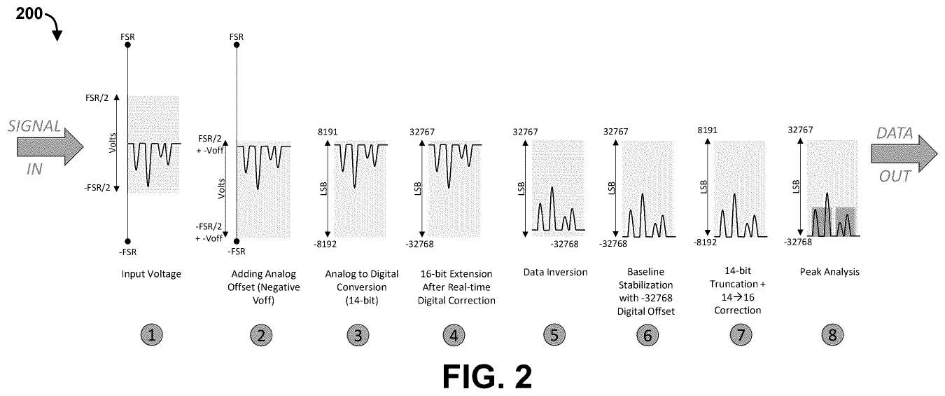

FIG. 2 is a diagram showing an example data flow in a digitizer (e.g., digitizer 114b in FIG. 1), according to some embodiments. As shown in FIG. 2, an analog signal (e.g., an input voltage signal) is received (at step 1) via a channel input connector of the digitizer (e.g., from a mass spectrometer, such as QTOF 120 in FIG. 1). The analog signal can correspond to or be generated based on an output from a TOF ion detector (e.g., TOF detector 120c in FIG. 1), which typically amplifies the signals of individual ions to detectable levels. At step 2, an analog offset “V_{off}” (e.g., having a negative value) is added to the received analog signal, and at step 3, an analog to digital conversion is performed, to produce digital data. At step 4, real-time digital correction is performed on the digital data. The real-time digital correction can include linearity and frequency response equalization, and results in corrected digital data (e.g., 16-bit data). At step 5, real-time signal processing is performed, which includes the following sequence of processes: data inversion, followed by baseline correction or stabilization (e.g., with digital offset), followed by correction based on a lookup table (LUT) (e.g., a custom/user-defined lookup table), optionally with bit-level truncation and/or correction), followed by one of real-time averaging (AVG), intensity thresholding (ZS1) alone, or RTB, depending on the mode of operation that the digitizer is in. The mode of operation of the digitizer can be user-selectable/modifiable. In addition, the LUT contents may user-selectable/modifiable/reprogrammable, for example based on one or more detector linearity correction algorithms. As shown in FIG. 2, RTB may be performed after sampling, baseline correction, and LUT correction. In some implementations, RTB is performed after all other operations on single scans have been performed.

FIGS. 3A-3B show plots illustrating RTB, according to some embodiments. More specifically, FIG. 3A shows a sequence of sets/plots of scan data (e.g., received at a digitizer, such as digitizer 114b of FIG. 1) associated with a plurality of scans performed by the TOF analyzer, and FIG. 3B shows a more detailed view of inset 300B of FIG. 3A. The sets/plots of scan data are separated by “triggers” (represented by vertical dashed lines). As used herein, a “trigger” can refer to a signal (e.g., received at the digitizer and from, for example, QTOF 120 and/or PCA(s) 112 of FIG. 1) or other event that indicates a transition between sets of scan data and/or scan data capture periods, and a “trigger marker” can refer to a representation (e.g., a label) in the data stream of an occurrence of an individual trigger. As can be seen in FIGS. 3A-3B, between each pair of adjacent triggers is a box that includes a cropped waveform that is associated with a data record (“ADC record”). Each ADC record can also include a plurality of gate records (optionally defined by a gating signal being passed, e.g., from the PCA(s) 112 to the digitizer 114b in FIG. 1) that coincide with the waveforms crossing above a threshold (e.g., a predefined intensity threshold) and are associated with peaks of the associated cropped waveforms. A dashed line representing a predefined intensity threshold is superimposed on one of the plots of FIG. 3B.

FIG. 3A also shows examples of summed (or “binned”) scan data (“Sum Scan 1,” “Sum Scan 2,” “Sum Scan 3”) generated (e.g., during RTB) by adding together the indicated sets/plots of scan data. Sum Scan 1 represents a summation of the scan data (e.g., adjacent scan data) between triggers 1 and 5, Sum Scan 2 represents a summation of the scan data (e.g., adjacent scan data) between triggers 5 and 10, and Sum Scan 3 represents a summation of the scan data (e.g., adjacent scan data) between triggers 10 and 15. Stated another way, Sum Scan 1 represents a summation of the cropped waveforms between triggers 1 and 5, Sum Scan 2 represents a summation of the cropped waveforms between triggers 5 and 10, and Sum Scan 3 represents a summation of the cropped waveforms between triggers 10 and 15. After Sum Scans 1-3 (“first summed scan data”) are generated, intensity thresholding may be applied to the Sum Scans 1-3 based on the threshold, to produce reduced associated data sets/plots (“second summed scan data”).

In some implementations, a number of triggers or a number of sets/plots of scan data (also referred to herein as a “binning number”) to include in a given summation/binning step is predefined (e.g., by a user), and can have a value, for example, of between 2 and 64. Alternatively or in addition, a maximum trigger length may be predefined (e.g., by a user), referring to a maximum number of data samples that can occur between sequential triggers, corresponding to the length of time data is recorded per trigger. The maximum trigger length can be, for example, 1,000,000 samples, which, for the example of a 2 GHz acquisition rate, would be a period of 500 μs.

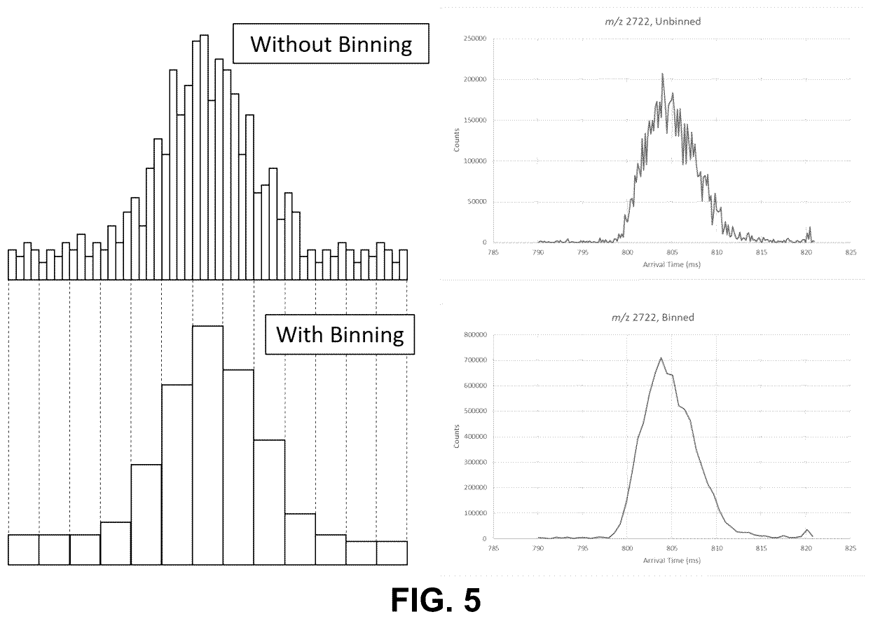

To facilitate comprehension of FIGS. 4-5, multiple nested timeframes may be considered. A first timeframe, shown in FIG. 4, relates to mass spectrum TOF, and a plot within this timeframe may be referred to as a “scan” or “spectrum.” A second timeframe relates to ion mobility separation, during which a large number (e.g., 5,000-10,000) of mass spectra are combined into a “frame.” Stated another way, as used herein, a “frame” can refer to a single scan through the ion mobility dimension, across a (optionally predefined) number of spectra. A third timeframe relates to liquid chromatography (LC) separation, which can be measured in terms of the frames. A chromatogram can be generated based on the signal variation that is observed across frames.

FIG. 4 is a diagram 400 showing how signal and noise are impacted by RTB, according to some embodiments. As shown in FIG. 4, each of two single/individual raw scans (left side of FIG. 4) includes data signals (signals A and B) and random noise signals, along with a horizontal dashed line indicating a predefined intensity threshold. When the two single scans are combined/summed/binned to produce the “RTB scan” (right side of FIG. 4), each of signal A and signal B is “boosted”/increased in magnitude, whereas the random noise is not, due to the fact that true signals are likely to overlap in subsequent scans while random noise does not. As a result, the signal to noise ratio is improved by the combining/summing/binning step of RTB. If the same predefined intensity threshold shown in the single scans is subsequently applied to the RTB scan, signal B—which would not otherwise have been detectable (e.g., in ZS mode)—is above the predefined intensity threshold and will be recorded in RTB mode. Thus, the sensitivity of the mass spectrometry signal is improved by RTB. Accordingly, the threshold in RTB may be set at a lower value, relatively speaking, than would have been used in ZS mode. If more single/individual raw scans are combined/summed/binned using RTB, additional smaller signals (which correspond to true/“real” ion TOF peaks, as opposed to noise) may be detected. Optionally, a “normalization” step is performed prior to applying the predefined intensity threshold, whereby the summed data and predefined intensity threshold are normalized/rescaled with respect to a full-scale intensity. In some such implementations, the predefined intensity threshold may be reduced by a factor of \sqrt{N} as the number of averages increases.

FIG. 5 is a diagram demonstrating the impact of real-time binning on ion mobility plots of counts versus arrival time, according to some embodiments. As can be observed in FIG. 5, ion mobility data without binning (the upper histogram and upper mobilogram) is more granular/choppier, whereas ion mobility data with binning (the lower histogram and lower mobilogram) is smoother and thus associated with a smaller data file, because fewer data points are required to describe the signal. As such, one or more RTB or binning methods described herein, when applied to ion mobility data, can result in reduced computational loads, reduced memory usage, and faster/more efficient processing than without RTB. As can be observed from FIG. 4 (depicting binning with respect to mass spectral sensitivity) and FIG. 5 (depicting binning with respect to ion mobility peak quality), binning provides benefits in both aspects.

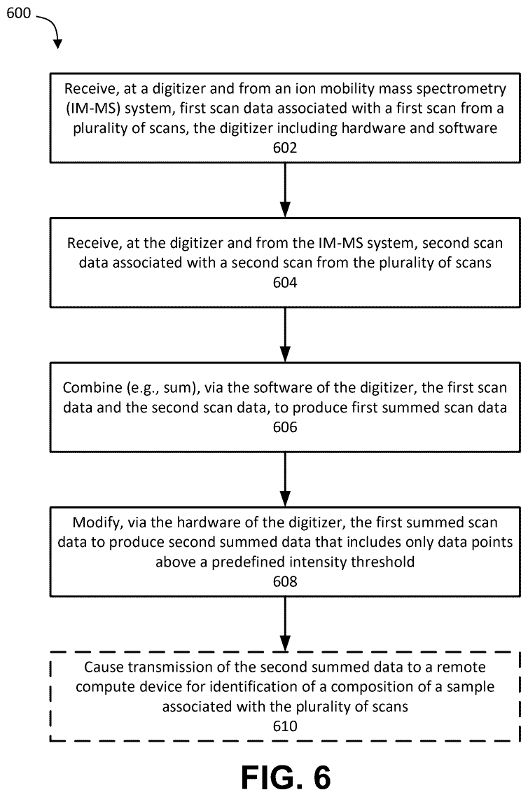

FIG. 6 is a flow diagram showing a first method for performing real-time binning of spectrometer data, according to some embodiments. The method 600 can be performed/implemented, for example, by system 100 depicted in FIG. 1. As shown in FIG. 6, the method 600 includes receiving, at 602, at a digitizer and from an ion mobility mass spectrometry (IM-MS) system, first scan data associated with a first scan from a plurality of scans, the digitizer including hardware (e.g., optionally including a field-programmable gate array (FPGA)) and software (optionally including firmware). The method also includes receiving, at 604, at the digitizer and from the IM-MS system, second scan data associated with a second scan from the plurality of scans. The method also includes summing or otherwise combining, at 606 (e.g., via the software of the digitizer, the first scan data and the second scan data, to produce first summed scan data. The method also includes modifying, at 608, via the hardware of the digitizer, the first summed scan data (e.g., based on a predefined intensity threshold) to produce second summed data that includes only data points above a predefined intensity threshold, e.g., the first summed scan data can be filtered to remove all data points below the predefined intensity threshold. The method also optionally includes causing transmission, at 610, of the second summed data to a remote compute device for identification of a composition of a sample associated with the plurality of scans.

In some implementations, the method 600 also includes modifying at least one of the first scan data or the second scan data based on a look-up table (e.g., a user-defined look-up table), via the digitizer and prior to the summing. Optionally, the method also includes modifying contents of the look-up table based on at least one detector linearity correction algorithm. Alternatively or in addition, the predefined intensity threshold may be adjusted based on a normalization factor and/or a number of scans binned.

In some implementations, the method also includes at least one of: (1) applying data inversion to the first scan data and the second scan data, via the digitizer and prior to the summing; (2) performing baseline correction of the first scan data and the second scan data, via the digitizer and prior to the summing; or (3) modifying at least one of the first scan data and the second scan data based on a look-up table, e.g., via the digitizer and prior to the summing.

The look-up table may be a user-defined look-up table. Optionally, contents of the look-up table are modified based on at least one detector linearity correction algorithm.

In some implementations, the second summed data includes data having a first signal-to-noise ratio that is higher than a second signal-to-noise ratio that would be obtained by performing the modifying without the preceding summing. Stated another way, the first signal-to-noise ratio can be higher than a second signal-to-noise ratio that would result from a method that does not perform RTB (e.g., that does not perform a combining/summing step, but that does perform a modification such as intensity thresholding).

In some implementations, the second summed data includes data having a first sensitivity that is higher than a second sensitivity that would be obtained by performing the modifying without the summing or by performing the summing without the modifying. Stated another way, the first sensitivity can be higher than a sensitivity that would result from a method that does not perform RTB (e.g., that does not perform a combining/summing step, but that does perform a modification such as intensity thresholding; or that does not perform a modification such as intensity thresholding, but that does perform a combining/summing step).

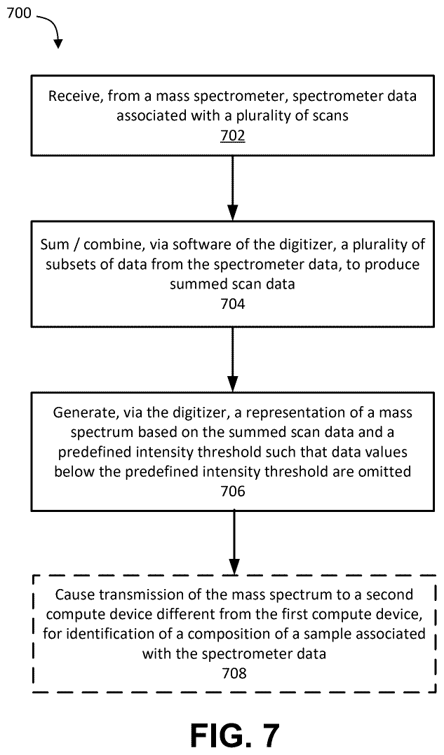

FIG. 7 is a flow diagram showing a second method for performing real-time data binning of spectrometer data, according to some embodiments. The method 700 can be performed/implemented, for example, by system 100 depicted in FIG. 1. As shown in FIG. 7, the method 700 includes receiving, at 702, at a first compute device (e.g., a digitizer) and from a mass spectrometer, spectrometer data associated with a plurality of scans. The method also includes summing or otherwise combining, at 704 (e.g., via software of the digitizer), a plurality of subsets of data from the spectrometer data, to produce summed scan data. The method also optionally includes generating, at 706 and via the digitizer, a representation of a mass spectrum based on the summed scan data and a predefined intensity threshold, such that data values below the predefined intensity threshold are omitted. The method also includes causing transmission, at 708, of the representation of the mass spectrum to a second compute device different from the first compute device, for identification of a composition of a sample associated with the spectrometer data. A number of subsets of data in the plurality of subsets of data may be selected based on a predefined binning number. The generation of the mass spectrum can be performed at least in part using hardware (e.g., one or more FPGAs) of the digitizer. In some implementations, the method 700 also includes identifying the plurality of subsets of data based on at least one trigger marker of the spectrometer data.

In some implementations, a number of subsets of data in the plurality of subsets of data is selected based on a predefined binning number.

In some implementations, the first compute device includes a digitizer, and the generation of the representation of the mass spectrum is performed at least in part using hardware of the digitizer. The hardware can include a field-programmable gate array (FPGA).

In some implementations, the instructions also include instructions to cause the processor to identify the plurality of subsets of data based on at least one trigger marker of the spectrometer data.

In some embodiments, an apparatus for performing real-time data binning of spectrometer data includes an ion mobility separation device coupled to a mass spectrometer of an IM-MS system, and a first compute device operably coupled to the mass spectrometer. At least one circuit board assembly of the IM-MS system is configured to perform ion mobility separation. The compute device includes a processor and a memory storing instructions that, when executed by the processor, cause the processor to receive, from the mass spectrometer, spectrometer data associated with a plurality of scans. The memory also stores instructions to cause the processor to generate a sum of a plurality of subsets of data from the spectrometer data, to produce summed scan data. A number of subsets of data in the plurality of subsets of data can be selected based on a predefined binning number. The memory also stores instructions to cause the processor to generate a representation of a mass spectrum based on the summed scan data and a predefined intensity threshold such that data values below the predefined intensity threshold are omitted. The memory also stores instructions to cause the processor to cause transmission of the representation of the mass spectrum to a second compute device different from the first compute device, for identification of a composition of a sample associated with the spectrometer data. The compute device can include a digitizer, and the instructions to generate the mass spectrum can include instructions to generate the mass spectrum at least in part using hardware (e.g., a field-programmable gate array (FPGA)) of the digitizer. The instructions can also include instructions to cause the processor to identify the plurality of subsets of data based on at least one trigger marker of the spectrometer data.

In some embodiments, a non-transitory processor-readable medium stores instructions that, when executed by a processor, cause the processor to receive, from a mass spectrometer of an IM-MS system, spectrometer data associated with a plurality of scans. The non-transitory processor-readable medium also stores instructions that, when executed by a processor, cause the processor to combine a plurality of subsets of data from the spectrometer data, to produce summed scan data. The non-transitory processor-readable medium also stores instructions that, when executed by a processor, cause the processor to identify a representation of a mass spectrum based on the summed scan data and a predefined intensity threshold. The non-transitory processor-readable medium optionally also stores instructions that, when executed by a processor, cause the processor to cause transmission of the representation of the mass spectrum for identification of a composition of a sample associated with the spectrometer data.

In some implementations, a number of subsets of data in the plurality of subsets of data can be selected based on a predefined binning number.

In some implementations, a number of subsets of data in the plurality of subsets of data is selected based on a binning number that may or may not be predefined initially, but that can be dynamically varied/adjusted over time, for example based on one or more (optionally user-defined) parameters such as, but not limited to, experimental parameter(s) and/or data readout(s).

In some implementations, the non-transitory processor-readable medium can also store instructions that, when executed by a processor, cause the processor to modify the spectrometer data, via the digitizer and based on a look-up table, prior to the summing.

In some implementations, the non-transitory processor-readable medium can also store instructions that, when executed by a processor, cause the processor to modify contents of the look-up table based on at least one detector linearity correction algorithm.

All combinations of the foregoing concepts and additional concepts discussed here within (provided such concepts are not mutually inconsistent) are contemplated as being part of the subject matter disclosed herein. The terminology explicitly employed herein that also may appear in any disclosure incorporated by reference should be accorded a meaning most consistent with the particular concepts disclosed herein.

The drawings are primarily for illustrative purposes, and are not intended to limit the scope of the subject matter described herein. The drawings are not necessarily to scale; in some instances, various aspects of the subject matter disclosed herein may be shown exaggerated or enlarged in the drawings to facilitate an understanding of different features. In the drawings, like reference characters generally refer to like features (e.g., functionally similar and/or structurally similar elements).

The entirety of this application (including the Cover Page, Title, Headings, Background, Summary, Brief Description of the Drawings, Detailed Description, Embodiments, Abstract, Figures, Appendices, and otherwise) shows, by way of illustration, various embodiments in which the embodiments may be practiced. The advantages and features of the application are of a representative sample of embodiments only, and are not exhaustive and/or exclusive. Rather, they are presented to assist in understanding and teach the embodiments, and are not representative of all embodiments. As such, certain aspects of the disclosure have not been discussed herein. That alternate embodiments may not have been presented for a specific portion of the innovations or that further undescribed alternate embodiments may be available for a portion is not to be considered to exclude such alternate embodiments from the scope of the disclosure. It will be appreciated that many of those undescribed embodiments incorporate the same principles of the innovations and others are equivalent. Thus, it is to be understood that other embodiments may be utilized and functional, logical, operational, organizational, structural and/or topological modifications may be made without departing from the scope and/or spirit of the disclosure. As such, all examples and/or embodiments are deemed to be non-limiting throughout this disclosure.

Also, no inference should be drawn regarding those embodiments discussed herein relative to those not discussed herein other than it is as such for purposes of reducing space and repetition. For instance, it is to be understood that the logical and/or topological structure of any combination of any program components (a component collection), other components and/or any present feature sets as described in the figures and/or throughout are not limited to a fixed operating order and/or arrangement, but rather, any disclosed order is exemplary and all equivalents, regardless of order, are contemplated by the disclosure.

The term “automatically” is used herein to modify actions that occur without direct input or prompting by an external source such as a user. Automatically occurring actions can occur periodically, sporadically, in response to a detected event (e.g., a user logging in), or according to a predetermined schedule.

The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

The term “processor” should be interpreted broadly to encompass a general purpose processor, a central processing unit (CPU), a microprocessor, a digital signal processor (DSP), a controller, a microcontroller, a state machine and so forth. Under some circumstances, a “processor” may refer to an application specific integrated circuit (ASIC), a programmable logic device (PLD), a field programmable gate array (FPGA), etc. The term “processor” may refer to a combination of processing devices, e.g., a combination of a DSP and a microprocessor, a plurality of microprocessors, one or more microprocessors in conjunction with a DSP core or any other such configuration.

The term “memory” should be interpreted broadly to encompass any electronic component capable of storing electronic information. The term memory may refer to various types of processor-readable media such as random access memory (RAM), read-only memory (ROM), non-volatile random access memory (NVRAM), programmable read-only memory (PROM), erasable programmable read only memory (EPROM), electrically erasable PROM (EEPROM), flash memory, magnetic or optical data storage, registers, etc. Memory is said to be in electronic communication with a processor if the processor can read information from and/or write information to the memory. Memory that is integral to a processor is in electronic communication with the processor.

The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may comprise a single computer-readable statement or many computer-readable statements.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Some embodiments and/or methods described herein can be performed by software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

Various concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

In addition, the disclosure may include other innovations not presently described. Applicant reserves all rights in such innovations, including the right to embodiment such innovations, file additional applications, continuations, continuations-in-part, divisionals, and/or the like thereof. As such, it should be understood that advantages, embodiments, examples, functional, features, logical, operational, organizational, structural, topological, and/or other aspects of the disclosure are not to be considered limitations on the disclosure as defined by the embodiments or limitations on equivalents to the embodiments. Depending on the particular desires and/or characteristics of an individual and/or enterprise user, database configuration and/or relational model, data type, data transmission and/or network framework, syntax structure, and/or the like, various embodiments of the technology disclosed herein may be implemented in a manner that enables a great deal of flexibility and customization as described herein.

All definitions, as defined and used herein, should be understood to control over dictionary definitions, definitions in documents incorporated by reference, and/or ordinary meanings of the defined terms.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method, comprising:
receiving, at a digitizer and from an ion mobility mass spectrometry (IM-MS) system, first scan data associated with a first scan from a plurality of scans, the digitizer including hardware and software;
receiving, at the digitizer and from the IM-MS system, second scan data associated with a second scan from the plurality of scans;
combining, via the software of the digitizer, the first scan data and the second scan data, to produce first summed scan data; and
modifying, via the hardware of the digitizer, the first summed scan data to produce second summed data that includes only data points above a predefined intensity threshold.

2. The method of claim 1, wherein the combining includes summing.

3. The method of claim 1, further comprising at least one of:
applying data inversion to the first scan data and the second scan data, via the digitizer and prior to the summing;
performing baseline correction of the first scan data and the second scan data, via the digitizer and prior to the summing; or
modifying the first scan data and the second scan data based on a look-up table, via the digitizer and prior to the summing.

4. The method of claim 1, further comprising modifying at least one of the first scan data or the second scan data based on a look-up table, via the digitizer and prior to the summing.

5. The method of claim 4, wherein the look-up table is a user-defined look-up table.

6. The method of claim 4, further comprising modifying contents of the look-up table based on at least one detector linearity correction algorithm.

7. The method of claim 1, wherein the software includes firmware.

8. The method of claim 1, wherein the hardware includes a field-programmable gate array (FPGA).

9. The method of claim 1, wherein the second summed data includes data having a first signal-to-noise ratio that is higher than a second signal-to-noise ratio that would be obtained by performing the modifying without the preceding summing.

10. The method of claim 1, wherein the second summed data includes data having a first sensitivity that is higher than a second sensitivity that would be obtained by performing the modifying without the summing or by performing the summing without the modifying.

11. An apparatus, comprising:
an ion mobility mass spectrometry (IM-MS) system including an ion mobility separation device; and
a first compute device operably coupled to the IM-MS system, the compute device including a processor and a memory storing instructions that, when executed by the processor, cause the processor to:
receive, from the IM-MS system, spectrometer data associated with a plurality of scans;
generate a sum of a plurality of subsets of data from the spectrometer data, to produce summed scan data;
generate a representation of a mass spectrum based on the summed scan data and a predefined intensity threshold such that data values below the predefined intensity threshold are omitted; and
cause transmission of the representation of the mass spectrum to a second compute device different from the first compute device.

12. The apparatus of claim 11, wherein a number of subsets of data in the plurality of subsets of data is selected based on a predefined binning number.

13. The apparatus of claim 11, wherein the compute device includes a digitizer, and the instructions to generate the mass spectrum include instructions to generate the mass spectrum at least in part using hardware of the digitizer.

14. The apparatus of claim 13, wherein the hardware includes a field-programmable gate array (FPGA).

15. The apparatus of claim 11, wherein the instructions further include instructions to cause the processor to identify the plurality of subsets of data based on at least one trigger marker of the spectrometer data.

16. A non-transitory processor-readable medium storing instructions that, when executed by a processor, cause the processor to:
receive, from a mass spectrometer of an ion mobility mass spectrometry (IM-MS) system, spectrometer data associated with a plurality of scans;
combine a plurality of subsets of data from the spectrometer data, to produce summed scan data; and
generate a representation of a mass spectrum based on the summed scan data and a predefined intensity threshold, the mass spectrum including only data points having values greater than the predefined intensity threshold.

17. The non-transitory processor-readable medium of claim 16, wherein a number of subsets of data in the plurality of subsets of data is selected based on a predefined binning number.

18. The non-transitory processor-readable medium of claim 16, further storing instructions that, when executed by the processor, cause the processor to modify the spectrometer data, via the digitizer and based on a look-up table, prior to the summing.

19. The non-transitory processor-readable medium of claim 18, further storing instructions that, when executed by the processor, cause the processor to modify contents of the look-up table based on at least one detector linearity correction algorithm.
